(19) United States
(12) Patent Application Publication
(10) Pub. No.: US 2014/0142490 A1
(43) Pub. Date: May 22, 2014

(54) SELF-ADHESIVE WOUND CARE PRODUCT

(71) Applicant: Ulf Johannison, Landvetter (SE)

(72) Inventor: Ulf Johannison, Landvetter (SE)

(73) Assignee: MÖLNLYCKE HEALTH CARE AB, Göteborg (SE)

(21) Appl. No.: 14/127,859

(22) PCT Filed: Jul. 2, 2012
(86) PCT No.: PCT/SE2012/050762

§ 371 Date: Dec. 19, 2013

Related U.S. Application Data
(60) Provisional application No. 61/505,566, filed on Jul. 8, 2011.

(30) Foreign Application Priority Data
Jul. 8, 2011 (SE) 1150653-2

(51) Int. Cl. A61F 13/02

(52) U.S. Cl. CPC A61F 13/0256
USPC 602/54

(57) ABSTRACT

A self-adhesive wound care product, including a backing layer having a self-adhesive coating, preferably a silicone-gel adhesive coating, the backing layer of the product comprising a first area in which the adhesive coating has a first thickness, and a second area along at least a portion of the edges of the product. The second area has an adhesive coating having a thickness, which is less than the first thickness and which gradually decreases in a direction towards the edge of the product, from a thickness which is substantially equal to said first thickness to a thickness of 0-0.05 mm; or the second area is substantially free from adhesive coating.

### TECHNICAL FIELD

The present invention relates to a self-adhesive wound care product.

### BACKGROUND

Self-adhesive wound care products or dressings are frequently used in wound care. Such wound care products typically include a flexible backing layer having an adhesive coating. Examples of wound care products of this type are given in WO2006075950 and EP633757.

Adhesive wound care products often have a tendency to start rolling in the edges, due to the friction between the adhesive dressing and materials, such as clothes or bed clothing, that the adhesive dressing contact. This is of course undesired, since it may lead to the wound care product being uncomfortable to wear, and to lost function of the product as it may not cover the wound properly.

It would be an advantage to provide adhesive wound care products that have an improved stay on ability, so that they do not roll up in the edge.

### SUMMARY OF THE INVENTION

According to the invention an adhesive dressing is provided, which has improved capability to remain attached to the skin at the outermost edges. The present invention thus relates to a self-adhesive wound care product, comprising a backing layer having a self-adhesive coating, preferably a silicone-gel adhesive coating, wherein the backing layer of the product comprises a first area in which the adhesive coating has a first thickness, and a second area along at least a portion of the edges of the product. The second area has an adhesive coating having a thickness, which is less that said first thickness and which gradually decreases in a direction towards the edge of the product, from a thickness which is substantially equal to said first thickness to a thickness of substantially 0-0.05 mm; or the second area is substantially free from silicone-gel adhesive coating. Due to the absence of adhesive at the outermost end of the product, the risk of rolling up is substantially decreased. In the following the self-adhesive coating is referred to as the silicon-gel adhesive coating, although soft hotmelt adhesives may alternatively be used.

### DRAWINGS

FIG. 1 represents a cross-sectional view of an example of a wound care product of the invention;

FIG. 2A-2C is a series of schematic views illustrating cutting of a wound care product of the invention;

FIG. 3A illustrates a tool for cutting wound care products according to the invention;

FIG. 3B represents a cross-sectional view of an end portion of a wound care product, cut with the tool of FIG. 3A;

FIG. 4 schematically illustrates tools for cutting of wound care products of the invention,

FIG. 4A shows a RDC tool for cutting products;

FIG. 4B represents a cross-sectional view along the line A-A of the cutting edge of a reference tool;

FIGS. 4C-4E represent cross-sectional views along the line A-A of the cutting edge of tools for cutting wound care products according to the invention;

FIG. 5A represents a photograph of a conventional wound dressing;

FIGS. 5B-5D represents photographs of wound dressings according to the invention;

FIGS. 6A and 6B represents a cross-sectional view of another example of a wound care product of the invention.

FIGS. 7-8 represent graphical illustrations of the results of a wear tests.

In the drawings, corresponding elements are represented by the same reference numerals.

### DETAILED DESCRIPTION

The self-adhesive wound care product comprises a backing layer having a self-adhesive coating, preferably a silicone-gel adhesive coating, wherein the outermost edge has a very thin adhesive coating, or is substantially free from adhesive coating. Thereby, the outermost portion of the backing layer will have a very low, or preferably non-existent, adhesive ability, which means that if or when the outermost portion of the product is unintentionally lifted from the skin, it will not adhere to other surfaces, such as the clothes or bed clothing. It will also not adhere to the upper surface of the backing layer. Instead, the outermost portion will fall back onto the skin. This absence of strong adhering properties of the outermost portion will largely reduce the risk of rolling up of the edges of the wound care product. Adhesiveness can be determined by measuring the adherence force against steel according to ASTM D. In order to be regarded as being non-adhesive in the sense of the present invention the adherence force should be less than 0.1 N/25 mm measured according to ASTM D 3330. The self-adhesive wound care product of the present invention has improved stay-on ability, which means that the tendency of self-loosening is reduced.

The solution provided by this invention is particularly suitable when the wound care product is a ready-to-use product, such as pre-cut products which come in ready-to-use sizes, adhesive wound care tapes, film dressings and other ready for use dressings, and ostomy products, such as base plates. The self-adhesive coating of the wound care product may alternatively be an acrylic or rubber based soft hotmelt coating.

In a first portion of the wound care product the backing layer comprises a first area in which the silicone-gel adhesive coating has a first thickness. This first portion is typically in the centre of the product, and the thickness of the adhesive coating in this area is preferably 0.05-0.3 mm, since silicone-gel adhesive coatings have proved to give good adherence to the skin, while being thin enough to provide excellent flexibility. The silicone-gel adhesive coating thickness may also be expressed as 50-300 g/m² of silicone-gel adhesive.

In a second portion of the wound care product, the silicone-gel adhesive coating is gradually decreasing towards the outer edge, from a thickness substantially equal to the adhesive coating of the first portion, to a thickness of about 0-0.05 mm, preferably 0 mm. By providing a silicone-gel adhesive coating, which has such low thickness at its outermost edge, the outermost portion of the backing layer will not be adhesive. Alternatively, the silicone-gel adhesive coating is substantially absent in the second portion, such that the second area is not adhesive. This second portion comprises an area along at least a portion of the edges of the product, and is adjacent to the first area, mentioned above, or adjacent to an intermediate area, located between said first and second areas. The second area having decreasing, and at the outermost edge no adhesive coating, or no adhesive coating at all, preferably extends along substantially all edges of the product, which results in a product which has improved stay-on properties from all directions. In some cases, the outermost part of the first portion may have an adhesive coating thickness in the area that exceeds the adhesive coating thickness of the rest of the first portion. The reason for this may be that the cutting tool used in production of the wound care product forces a small amount of adhesive in a direction towards the centre of the product. However, the stay-on ability of the wound care product is not affected by this.

The wound care product may alternatively include at least three portions having different adhesive coating thicknesses. The first portion may then be as described above, and the second area may have a silicone-gel adhesive coating with gradually decreasing thickness, and in addition the product may further comprise a third portion, which is an area at the outermost edge, being substantially free from silicone-gel adhesive coating, to such an extent that the third area is not adhesive.

The area which is substantially free from silicone-gel adhesive coating preferably has a width of at least 0.5 mm, preferably at least 1 mm, and preferably up to 2 mm. This ensures both excellent adhesive properties of the wound care product, and excellent stay-on ability.

In all variants of the wound care product of this invention, the backing layer is a flexible layer, which can readily be applied to any part of the wearer's body. The backing layer is suitably a polymer film, preferably a polyurethane film, as such films are suitable for this purpose. The plastic film, in particular the polyurethane film, may have a thickness of less than 100 μm, preferably 10-50 μm, in order to allow the wound care product to adapt closely to the shape of the wearer's body. The backing layer may alternatively be a laminate, which could suitably include an elastic film made of for example polyurethane (PU), ethylene vinyl acetate (EVA) or thermoplastic elastomer (TPE). The laminate could also include a layer of nonwoven or textile material. When using a laminate it may be preferred that one of the laminate layers has a larger surface area than the other ones, so that a portion of the largest laminate layer extends outside of the layer(s) to which it is laminated, and forms the outermost non-adhesive portion. Such a wound care product may be manufactured similarly to an island dressing, having adhesive coating only on non absorbing wound pad and no adhesive coating on the outermost portion layer with the largest surface area. The layers of the laminate may be laminated by adhesive lamination, using for example hotmelt or acrylic glue, heat lamination or flame lamination.

As an alternative, a separate edge portion can be attached to the edge of the backing layer, said edge portion being non-adhesive and forming the outermost non-adhesive portion of the wound care product. The separate edge portion could be attached edge to edge to the backing layer, or be partially overlapping the backing layer. Such a separate edge portion may suitably be attached to the edge of the backing layer by means of welding or gluing. In this alternative, the backing layer having adhesive coating can be either a single layer backing layer or a laminate.

A carrier layer may also be provided on the backing layer, in order to facilitate handling of the wound care product. The function of the carrier layer is to support the plastic film and to provide steadiness, while applying the wound care product to the wearer's skin, whereupon the carrier layer is removed. Such carrier layer may be a plastic film of somewhat higher stiffness than the material used for the backing layer, or nonwoven material, flexible foam material, or paper, which advantageously is polyethylene-coated, or the like.

In order to achieve desired widths on the outermost edges, having no or almost no adhesive coating, there are certain parameters to take into consideration, such as web-material thickness, silicone thickness, and silicone stiffness.

The web-material thickness is often given by the product design. Sometimes a carrier layer is used. It is also common that the web-material thickness differs over the product area, due to the normal design of release liners, which may be folded and/or overlapping.

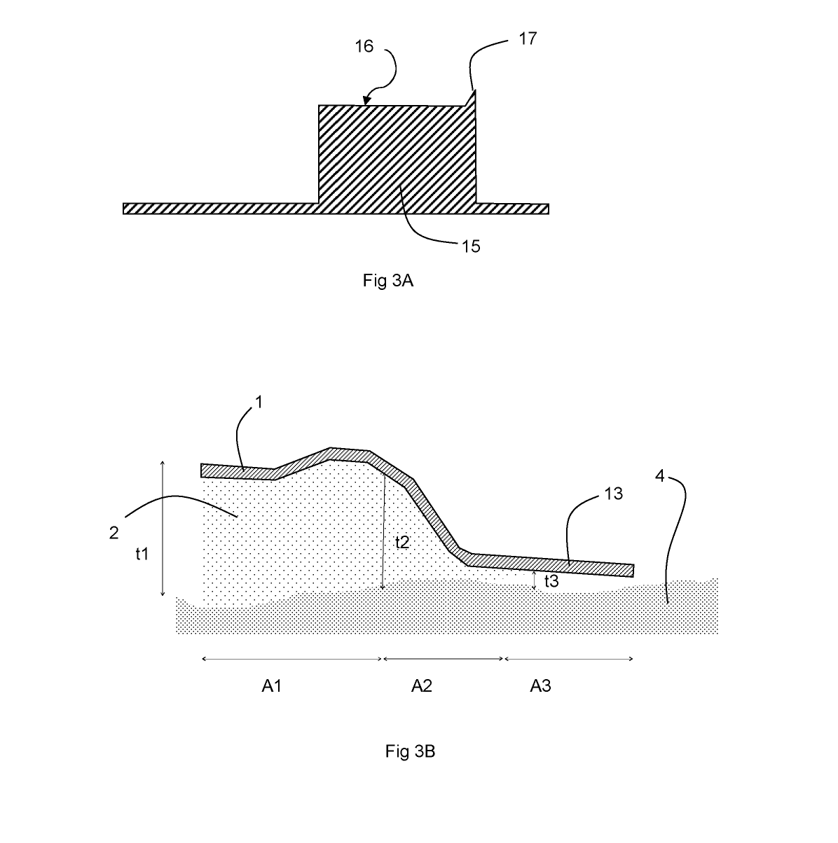

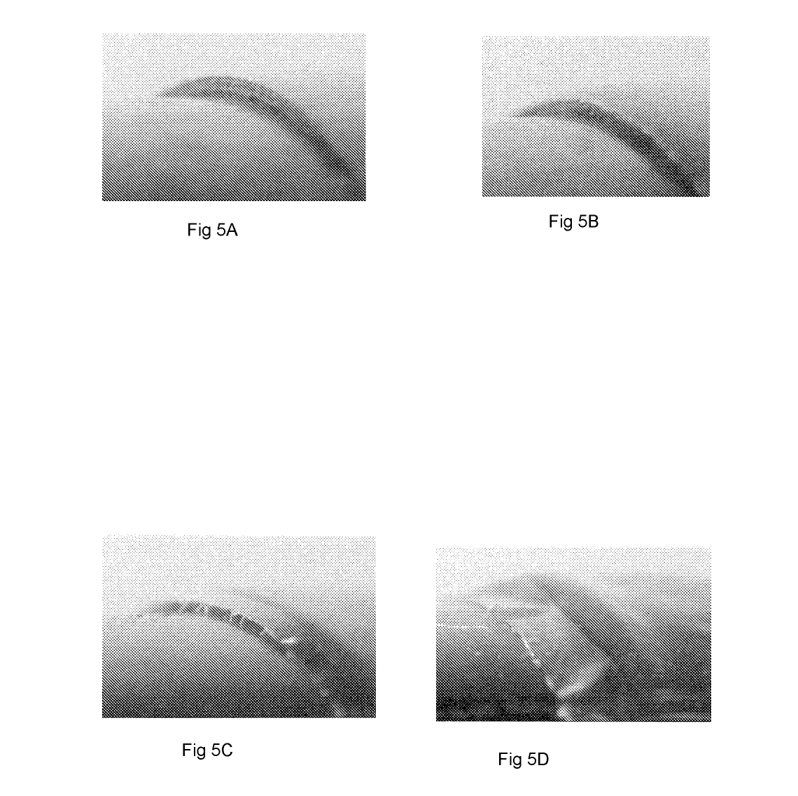

As to the silicone-gel adhesive thickness a high grammage (gsm) means that there is more material to be moved away from the edge. In order to obtain an attractive visual appearance it is desired that all the “extra material” would be pushed into the waste material outside the die-cut, but in reality some of the silicone is pushed into the product. This can be seen in FIGS. 3B, 5C and 5D, which are described below. Thereby, the product will include an intermediate area between the first and second areas, which intermediate area has an adhesive layer having a thickness, which is higher than the adhesive layer thickness of the first and second areas. This may contribute positively to the stay on ability, as the adhesive layer will be thicker.

Silicone stiffness also affects the resulting wound care product with regards to penetration. There is more memory in a stiff gel-material, which means that it is necessary to push the silicone-gel adhesive material further away from the edge, because it will slowly move back a little bit after punching (cutting). This phenomenon can take up to 48 h.

Of the above mentioned parameters, the web-material thickness gives the largest contribution to the end result. It has been found that the most powerful way of controlling the properties of the edges with decreasing or substantially absent silicone gel adhesive coating, is to adjust the angle of the cutting edge (α). When a high web material thickness is used a higher angle is needed, because otherwise the gel will be pushed too far from the edge.

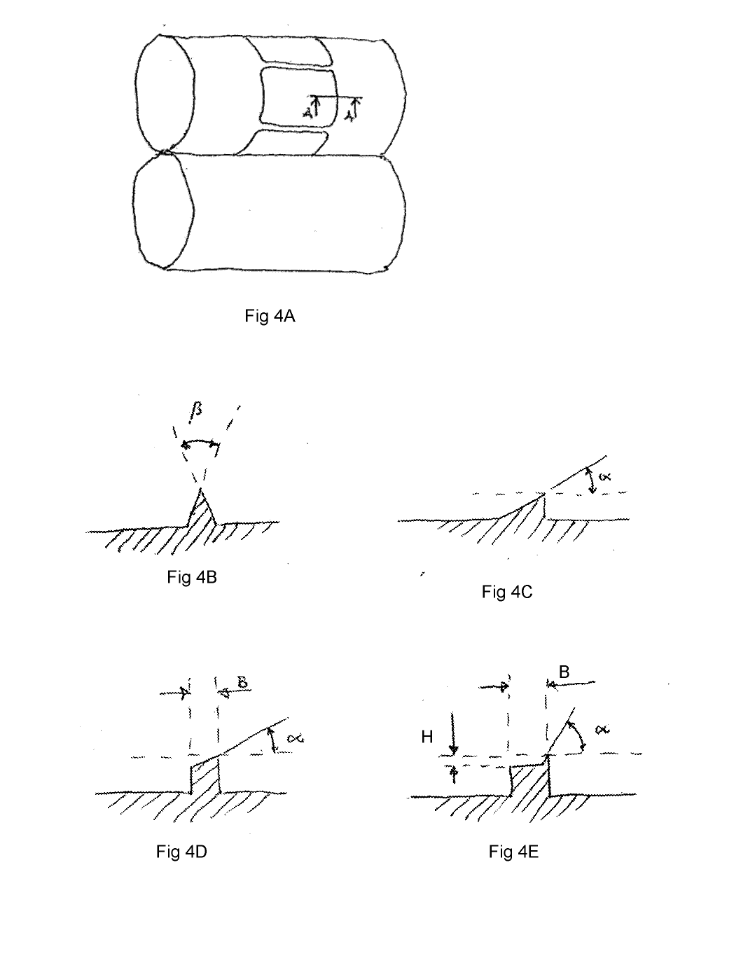

The self-adhesive wound care product of this invention may be obtained by a process in which single wound care products are cut out from a blank sheet comprising a backing layer, a silicone-gel adhesive layer and a release layer, preferably by Rotary Die Cutting (RDC). Any other suitable die cutting process is of course applicable, such as flatbed cutting. By using a cutting tool having a chamfered cutting edge, the gradually decreasing silicone-gel adhesive coating thickness is obtained. A flat surface cutting tool with a sharp peripheral protruding cutting edge can result in an edge portion of the wound care product which is substantially free from silicone-gel adhesive. FIG. 4 schematically illustrates tools for cutting of wound care products of the invention. A plurality of cutting tools may be arranged on a cylinder of an RDC cutting apparatus, see FIG. 4A, where each cutting tool includes a cutting edge defining the circumference of the wound care product to be cut. FIG. 4B represents a cross-sectional view along the line A-A of the cutting edge of a reference tool, in which the cutting edge is formed between two inclined cutting tool surfaces, where the angle between these surfaces is β, typically being about 20°. FIGS. 4C-4E represent cross-sectional views along the line A-A of the cutting edge of tools for cutting wound care products according to the invention. It is to be noted that other cutting edge shapes may also be contemplated. In the cutting tool illustrated in FIG. 4C the cutting edge is formed between a cutting tool surface, which is perpendicular to the cylinder surface, and a cutting tool surface, which is inclined in an angle α relative to the cylinder surface. The angle α may be 5-25°, preferably 10-20°. The cutting edge illustrated in FIG. 4D is also formed between a cutting tool surface, which is perpendicular to the cylinder surface, and a cutting tool surface, which is inclined in an angle a relative to the cylinder surface, but in this case the cutting edge is arranged on a larger distance from the cylinder inclined surface by means of a section having rectangular cross-section, below the inclined surface, said rectangular section having a width B, which may be 1-3 mm. The angle α may be 5-25°, preferably 10-20°. The cutting edge shown in FIG. 4E also includes a section having rectangular cross-section, but has a small section with triangular cross-section at the outermost side edge, thus, forming a flat surface with a small cutting edge at the outer periphery at a distance H from the flat surface. The height H of the triangular cutting edge may be 0.05-0.5 mm. The rectangular section has a width B, which may be 1-3 mm. The cutting edge is formed between a cutting tool surface, which is perpendicular to the cylinder surface, and a cutting tool surface, which is inclined in an angle α relative to the cylinder surface. The angle α may be 55-80°, preferably 65-75°.

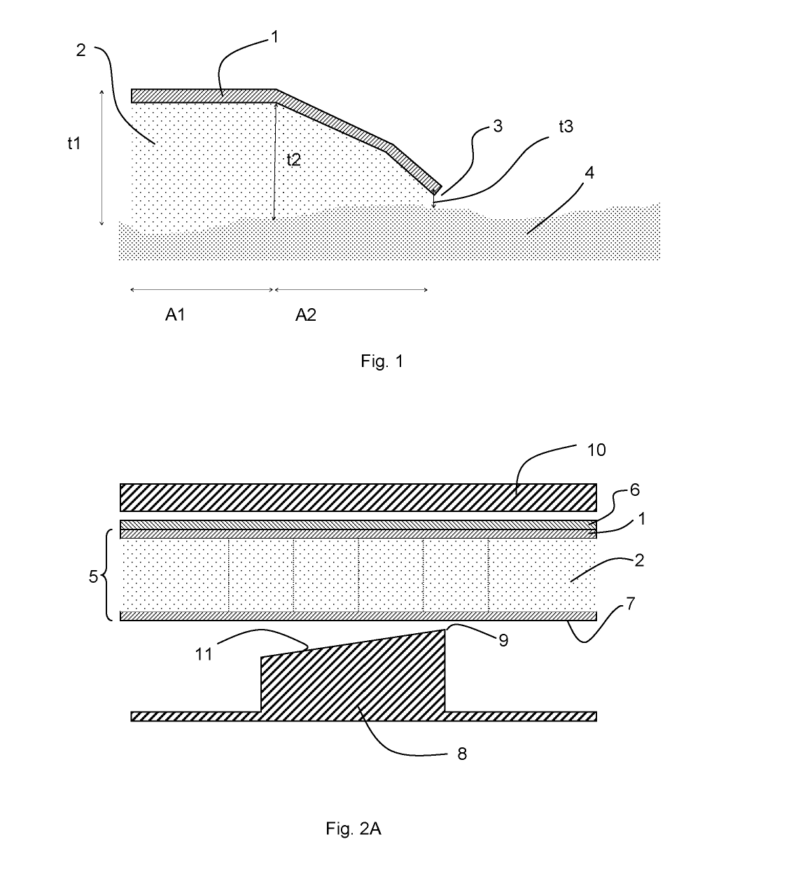

An example of a wound care product of the invention is illustrated in FIG. 1, as applied on the skin 4 of a wearer. The wound care product comprises a backing layer 1 having a self-adhesive silicone-gel adhesive coating 2. In the first area A1, the backing layer 1 has a self-adhesive silicone-gel adhesive coating having a thickness t1. In this example the second area A2 has a silicone-gel adhesive coating has a thickness that gradually decreases in the direction towards the edge 3 of the product, from a thickness t2 which is substantially equal to adhesive coating thickness t1 of the first area, to a thickness t3 of substantially 0-0.05 mm.

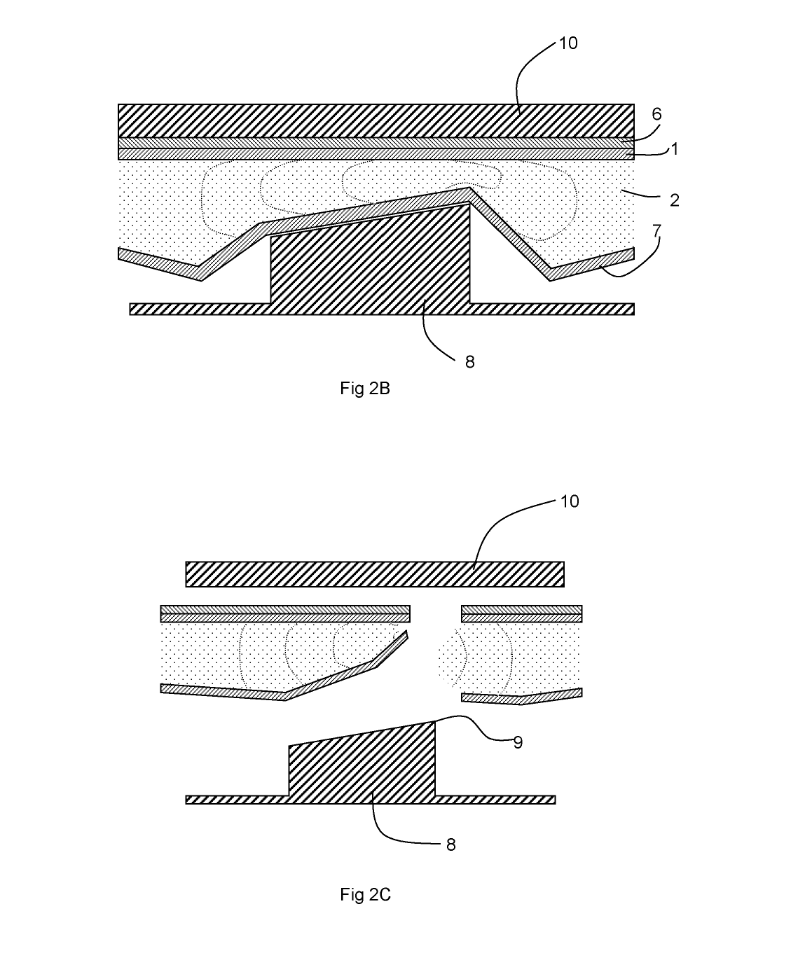

FIG. 2 illustrates how a wound care product of the invention can be obtained. FIG. 2A-2C is a series of schematic views illustrating how the wound care product can be cut. In the example shown in this Figure, the blank includes a carrier layer 6, a backing layer 1, a silicone-gel adhesive coating 2 and a release layer 7. In FIG. 2A the cutting tool 8 has not yet come into contact with the wound care product blank 5. The tool 8 has a chamfered pressing surface 11, which is inclined relative to the blank and to the anvil, and a cutting edge 9 at the end of the pressing surface. FIG. 2B shows how the tool 8 is pushed against the blank, so that the silicone-gel adhesive coating is pressed to the sides. At last, as shown in FIG. 2C, the edge 9 of the tool 8 will come into contact with the anvil 10, whereby the release layer and backing layer is cut, and the cutting tool 8 is then retracted. The thus produced wound care product, seen to the left in FIG. 2C, has now a gradually decreasing silicone-gel adhesive coating in the edge area, and is substantially free from adhesive at the outermost edge. When the release layer is removed and the wound care product is applied to the skin, the product will have the appearance as shown in FIG. 1.

FIG. 3A illustrates an alternative cutting tool 15 for cutting wound care products according to the invention. In this example, the pressing surface 16 is flat and substantially parallel to the blank and anvil, and the cutting edge 17 extends perpendicularly to the pressing surface, see also FIG. 4E, which shows a tool of the same design. Cutting with the tool 15 of FIG. 3A results in a wound care product having a third portion A3, i.e. an edge portion 13, which is substantially free from silicone-gel adhesive, which is illustrated in FIG. 3B. In this example, the adhesive thickness of the outermost part of the first portion (A1) exceeds the adhesive coating thickness t1 of the rest of the first portion as a result of the cutting with the tool 15. The adhesive coating thickness then gradually decreases towards the outer edges until it reaches a thickness t3, which is substantially zero, so that the outermost portion 13 of the backing is substantially non-adhesive.

FIG. 5 shows photographs of wound dressings cut with different tools from a 20 μm PU-film having 200 g/m² of silicone gel adhesive applied thereon. FIG. 5A shows a conventional wound dressing, cut with a Reference tool having the shape as shown in FIG. 4B, β=20°; and FIGS. 5B-5D show photographs of wound dressings according to the invention, cut with tools having a shape as shown in FIG. 4D, α=15° (FIG. 5B); FIG. 4D, α=10° (FIG. 5C); and FIG. 4E, α=70°, B=2 mm, H=0.1 mm (FIG. 5D), respectively. Wear tests have been performed in order to study how the gradually decreasing silicone-gel adhesive coating thickness or the absence silicone-gel adhesive coating affects the stay-on abilities of the wound care product.

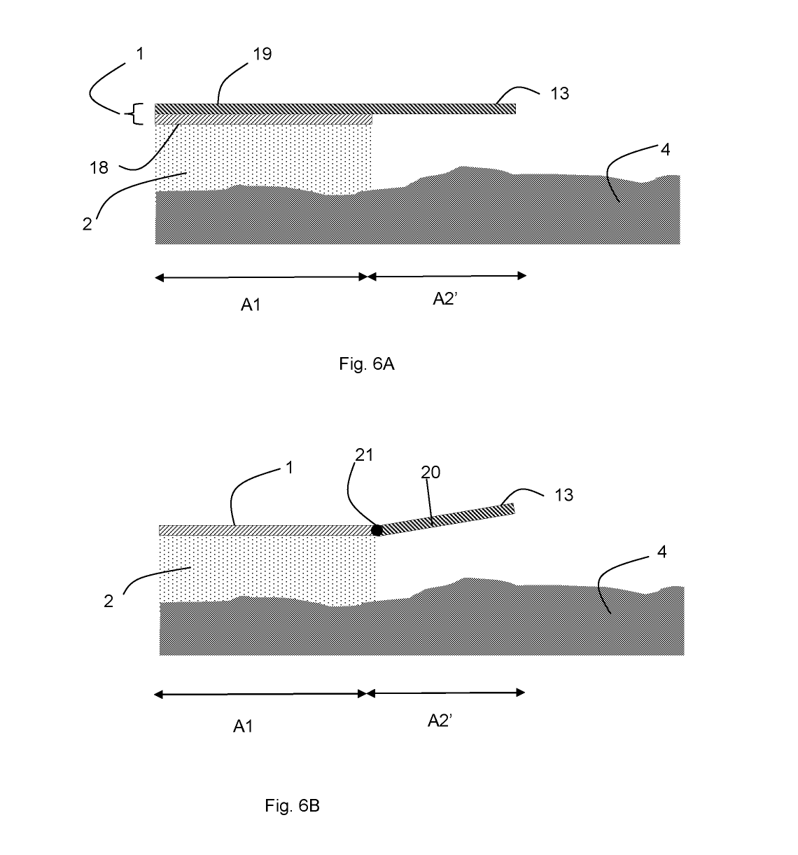

FIG. 6 illustrates alternative variants of the wound care product of the invention. In the variant shown in FIG. 6A the backing layer 1 is a laminate, where an outer layer 19 is laminated onto an inner layer 18, and the outer layer 19 has a larger surface area than the inner layer 18, so that the outermost portion of the outer layer 19 extends beyond the edge of the inner layer. The outermost edge portion 13 of the layer 19 is free from adhesive and constitutes a non-adhesive area A2′. In the variant shown in FIG. 6B a separate edge portion 20 is attached by a weld line 21 to the edge of a backing layer 1 having an adhesive coating 2, so that the separate edge portion 20, which is free from adhesive constitutes a non-adhesive area A2′.

### Wear Test

The purpose of the wear test was to evaluate different product edge cuts during use. The wound care products tested included a backing layer in the form of a film or a laminate, and an adhesive layer applied to the backing layer. In the case of wound care products having a film as backing layer, the film was a 20 μm siliconized PUR film, having a silicone-gel adhesive coating of 200 g/m² (Wear test 1), and in the case of wound care products having a laminate as backing layer, the laminate was a double-stick perforated 25 μm PU film having a silicone gel layer of 200 g/m², and being laminated to a non-perforated 20 μm PU film having a 20 g/m² acrylate adhesive layer (Wear test 2). The perforations amount to 13% of the surface area of the perforated PU film. The factor to evaluate was self-loosening, i.e. the opposite to stay-on-ability. Test products as shown in Table 2 were cut by a RDC tool, using dies as indicated in Table 1, and had a size of 6×6.5 cm².

TABLE 1

| Tool die used for | |
|---|---|
| cutting the test products | Cross-sectional shape of cutting edge |
| Ref A | FIG. 4B, β = 20° |
| 1 | FIG. 4D, α = 15° |
| 2 | FIG. 4E, α = 70°, B = 2 mm, H = 0.1 mm |
| Ref B | FIG. 4B, β = 20° |
| 3 | FIG. 4D, α = 10° |

TABLE 2

| | | RCD tool die | | | |
|---|---|---|---|---|---|
| | | used for | No of test | | Total No of |
| Wear | Test | cutting the | products per | No of | test products |
| test | product | test product | volunteer | volunteers | tested |
| 1 | Film Ref | Ref A | 4 | 6 | 24 |
| | Film 1 | 1 | 4 | 6 | 24 |
| | Film 2 | 2 | 4 | 6 | 24 |
| 2 | Laminate | Ref B | 6 | 6 | 36 |
| | Ref | | | | |
| | Laminate | 3 | 6 | 6 | 36 |
| | 1 | | | | |

The test products cut by tools Ref A and Ref B represent conventionally cut wound care products that do not have a gradually decreasing adhesive thickness towards the outer edges of the product. The test product Film 1 represents one example of the invention, where the thickness of the adhesive in the outermost area gradually decreases to approximately 0 mm (see also FIG. 2 and FIG. 5B), and the test product Film 2 represents another example of the invention where an area which is substantially free from adhesive coating is present at the outermost edges of the products. In this test the non-adhesive area of the Film 2 test product has a width of approximately 2 mm. The test product Laminate 1 has a gradually decreasing adhesive layer thickness, similar to the test product Film 1.

The tests were performed on healthy volunteers. The test products were attached on the back of each volunteer. It was carefully checked that test products were placed both with the machine direction (MD) vertically, and in random positions. The test products were attached by striking the product gently onto the skin after application, and it was verified that the edges of all test products were completely attached to the skin. The test products were worn for 72 hours, and the participants were allowed to exercise and to take a shower during these hours. All participants were instructed not to use any moisturizing cream or similar on the skin.

The products were visually inspected after 24, 48 and 72 hours by one appointed control person, in which inspection self-loosening of each test product from the skin was determined. In this context loosening includes any ever so small loosening of an edge or border.

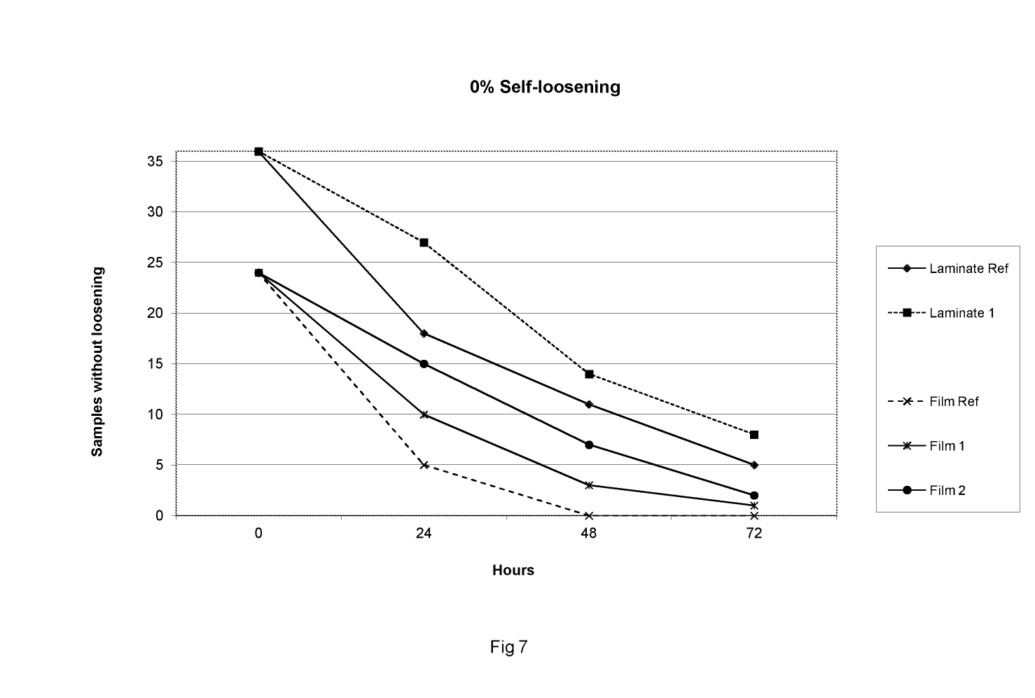

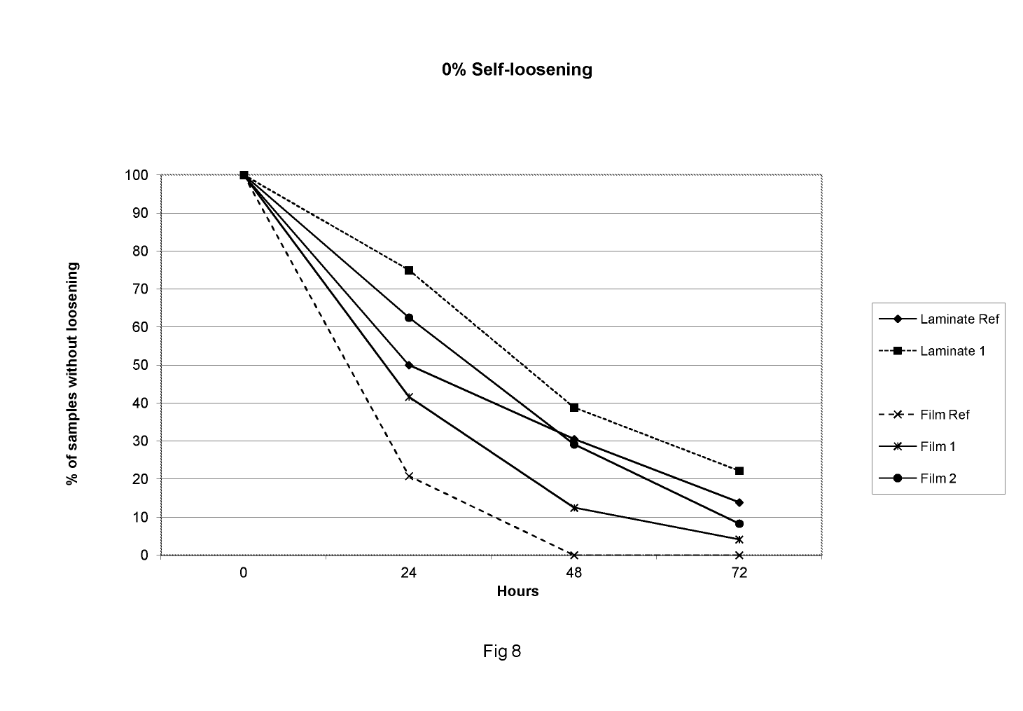

FIGS. 7-8 show the result of the wear tests, wherein FIG. 7 shows the number of test products that has intact adhesion to the skin, i.e. had not loosened, at 24, 48 and 72 hours, respectively, and FIG. 8 shows the same result expressed in percent. As can be seen the test products according to the invention have a decreased tendency to loosen at the edges, as compared to the conventionally cut test products, which are equally adhesive over their entire surface area. Thus, the wear tests clearly show that the wound care products according to the invention have an improved ability to remain attached to the skin at the outermost edges, and that this characteristic is even more pronounced when there is an outermost non-adhesive area substantially free from adhesive.

1-7. (canceled)

8. A self-adhesive wound care product, comprising a backing layer having a self-adhesive coating, wherein the backing layer comprises:
at least one edge;
a first area distal from said at least one edge in which said adhesive coating has a first thickness; and
a second area between said first area and said at least one edge, wherein said adhesive coating in said second area has a second thickness that gradually decreases in a direction toward said at least one edge from a thickness that is substantially equal to said first thickness to a thickness of 0.00 mm to 0.05 mm.

9. The self-adhesive wound care product of claim 8, wherein said second area extends along at least a portion of said at least one edge.

10. The self-adhesive wound care product of claim 8, wherein said backing layer comprises a plurality of edges, and wherein said second area extends along substantially all said edges.

11. The self-adhesive wound care product of claim 8, further comprising a third area between said second area and said at least one edge, wherein said third area extends along said at least one edge, and wherein said third area is substantially free from said adhesive coating.

12. The self-adhesive wound care product of claim 11, wherein said third area has a width of at least 0.5 mm.

13. The self-adhesive wound care product of claim 11, wherein said third area has a width of at least 1 mm.

14. The self-adhesive wound care product of claim 8, wherein said self-adhesive coating comprises a silicone gel.

15. The self-adhesive wound care product of claim 8, wherein said backing layer comprises a polymer film

16. The self-adhesive wound care product of claim 15, wherein said backing layer comprises a polyurethane film.

17. The self-adhesive wound care product of claim 8, wherein said first thickness of said adhesive coating is 0.05 to 0.3 mm.

18. The self-adhesive wound care product of claim 8, made by the steps of:
providing a sheet comprising a backing layer, an adhesive layer, and a release layer; and
cutting individual wound care products using a cutting tool having a chamfered edge.

19. A self-adhesive wound care product, comprising a backing layer having a self-adhesive coating, wherein the backing layer comprises:
at least one edge;
a first area distal from said at least one edge in which said adhesive coating has a first thickness; and
a second area extending along at least a portion of said at least one edge, wherein said second area is substantially free from said adhesive coating.

20. The self-adhesive wound care product of claim 19, wherein said backing layer comprises a plurality of edges, and wherein said second area extends along substantially all said edges.

21. The self-adhesive wound care product of claim 19, wherein said second area has a width of at least 0.5 mm.

22. The self-adhesive wound care product of claim 19, wherein said second area has a width of at least 1.0 mm.

23. The self-adhesive wound care product of claim 19, wherein said self-adhesive coating comprises a silicone gel.

24. The self-adhesive wound care product of claim 19, wherein said backing layer comprises a polymer film

25. The self-adhesive wound care product of claim 24, wherein said backing layer comprises a polyurethane film.

26. The self-adhesive wound care product of claim 19, wherein said first thickness of said adhesive coating is 0.05 to 0.3 mm.

27. The self-adhesive wound care product of claim 19, made by the steps of:
providing a sheet comprising a backing layer, an adhesive layer, and a release layer; and
cutting individual wound care products using a cutting tool having a chamfered edge.
